(19) United States
(12) Patent Application Publication
(10) Pub. No.: US 2017/0281936 A1
(43) Pub. Date: Oct. 5, 2017

(54) Convertible Implantable Stimulator

(71) Applicant: Boston Scientific Neuromodulation Corporation, Valencia, CA (US)

(72) Inventors: Daniel Aghassian, Glendale, CA (US); Ken William Mariash, JR., Los Angeles, CA (US); Jeffery Van Funderburk, Stevenson Ranch, CA (US); Zdzislaw Bernard Malinowski, Castaic, CA (US); Vuong Tuan Nguyen, Glendale, CA (US)

(21) Appl. No.: 15/472,095

(22) Filed: Mar. 28, 2017

Related U.S. Application Data
(60) Provisional application No. 62/317,059, filed on Apr. 1, 2016.

(51) Int. Cl. A61N 1/36

(52) U.S. Cl. CPC A61N 1/3605

(57) ABSTRACT

A convertible implantable stimulator that provides electrical stimulation therapy during an extended trial stimulation period (or permanently, if desired) in a fully implanted solution is disclosed. The convertible implantable stimulator preferably does not include an internal power supply and is therefore continuously powered by an external charger, such as a powering patch, in a first mode of operation. If the convertible implantable stimulator is determined to be effective and a patient desires more traditional stimulation therapy, a separate power supply module can subsequently be implanted and connected to the convertible implantable stimulator to provide power to the stimulator in a second mode of operation.

### CROSS-REFERENCE TO RELATED APPLICATIONS

This is a non-provisional of U.S. Provisional Patent Application Ser. No. 62/317,059, filed Apr. 1, 2016, to which priority is claimed, and which is incorporated herein by reference in its entirety.

### FIELD OF THE TECHNOLOGY

The present application relates to an implantable pulse generator (IPG) that is implantable through a routine procedure and which receives continuous operating power from an external power supply in a first mode of operation and from a separately implanted power supply module in a second mode of operation.

### INTRODUCTION

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and Deep Brain Stimulators (DBS) to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability with any Implantable Pulse Generator (IPG) or in any IPG system.

As shown in FIG. 1, a traditional SCS system includes an Implantable Pulse Generator (IPG) 10, which includes a biocompatible device case 12 formed of titanium for example. The case 12 typically holds the circuitry and battery 14 (FIG. 2) necessary for the IPG 10 to function, which battery 14 may be either rechargeable or primary (non-rechargeable) in nature. The IPG 10 is coupled to electrodes 16 via one or more electrode leads 18 (two of which are shown). The proximal ends of the leads 18 include electrode terminals 20 that are coupled to the IPG 10 at one or more connector blocks 22 fixed in a header 24, which can comprise an epoxy for example. Contacts in the connector blocks 22 make electrical contact with the electrode terminals 20, and communicate with the circuitry inside the case 12 via feedthrough pins 26 passing through a hermetic feedthrough 28 to allow such circuitry to provide stimulation to or monitor the various electrodes 16. In the illustrated system, there are sixteen electrodes 16 split between two leads 18, although the number of leads and electrodes is application specific and therefore can vary. In a traditional SCS application, two electrode leads 18 are typically implanted on the right and left side of the dura within the patient's spinal column.

As shown in FIG. 2, IPG 10 contains a charging coil 30 for wireless charging of the IPG's battery 14 using an external charging device 50, assuming that battery 14 is a rechargeable battery. If IPG 10 has a primary battery 14, charging coil 30 in the IPG 10 and external charger 50 can be eliminated. IPG 10 also contains a telemetry coil antenna 32 for wirelessly communicating data with an external controller device 40, which is explained further below. In other examples, antenna 32 can comprise a short-range RF antenna such as a slot, patch, or wire antenna. IPG 10 also contains control circuitry such as a microcontroller 34, and one or more Application Specific Integrated Circuit (ASICs) 36, which can be as described for example in U.S. Pat. No. 8,768,453. ASIC(s) 36 can include stimulation circuitry for providing stimulation pulses at one or more of the electrodes 16 and may also include telemetry modulation and demodulation circuitry for enabling bidirectional wireless communications at antenna 32, battery charging and protection circuitry coupleable to charging coil 30, DC-blocking capacitors in each of the current paths proceeding to the electrodes 16, etc. Components within the case 12 are integrated via a printed circuit board (PCB) 38.

FIG. 2 further shows the external components referenced above, which may be used to communicate with the IPG 10, in plan and cross section views. External controller 40 may be used to control and monitor the IPG 10 via a bidirectional wireless communication link 42 passing through a patient's tissue 5. For example, the external controller 40 may be used to provide or adjust a stimulation program for the IPG 10 to execute that provides stimulation to the patient. The stimulation program may specify a number of stimulation parameters, such as which electrodes are selected for stimulation; whether such active electrodes are to act as anodes or cathodes; and the amplitude (e.g., current), frequency, and duration of stimulation at the active electrodes, assuming such stimulation comprises stimulation pulses as is typical.

Communication on link 42 can occur via magnetic inductive coupling between a coil antenna 44 in the external controller 40 and the IPG 10's telemetry coil 32 as is well known. Typically, the magnetic field comprising link 42 is modulated, for example via Frequency Shift Keying (FSK) or the like, to encode transmitted data. For example, data telemetry via FSK can occur around a center frequency of fc=125 kHz, with a 129 kHz signal representing transmission of a logic ‘1’ bit and 121 kHz representing a logic ‘0’ bit. However, transcutaneous communications on link 42 need not be by magnetic induction, and may comprise short-range RF telemetry (e.g., Bluetooth, WiFi, Zigbee, MICS, etc.) if antennas 44 and 32 and their associated communication circuitry are so configured. The external controller 40 is generally similar to a cell phone and includes a hand-held, portable housing.

External charger 50 provides power to recharge the IPG's battery 14 should that battery be rechargeable. Such power transfer occurs by energizing a charging coil 54 in the external charger 50, which produces a magnetic field comprising transcutaneous link 52, which may occur with a different frequency (f2=80 kHz) than data communications on link 42. This magnetic field 52 energizes the charging coil 30 in the IPG 10, which is rectified, filtered, and used to recharge the battery 14. Link 52, like link 42, can be bidirectional to allow the IPG 10 to report status information back to the external charger 50, such as by using Load Shift Keying as is well-known. For example, once circuitry in the IPG 10 detects that the battery 14 is fully charged, it can cause charging coil 30 to signal that fact back to the external charger 50 so that charging can cease. Like the external controller 40, external charger 50 generally comprises a hand-holdable and portable housing.

External controller 40 and external charger 50 are described in further detail in U.S. Patent Application Publication 2015/0080982. Note also that the external controller 40 and external charger 50 can be partially or fully integrated into a single external system, such as disclosed in U.S. Pat. Nos. 8,335,569 and 8,498,716.

As described above, the electrical stimulation that the IPG 10 is capable of delivering is highly customizable with respect to selected electrodes, electrode current amplitude and polarity, pulse duration, pulse frequency, etc. Due to uncertainties in the location of electrodes with respect to neural targets, the physiological response of a patient to stimulation patterns, and the nature of the electrical environment within which the electrodes are positioned, it is challenging to determine the stimulation parameters that might provide effective stimulation therapy for a particular patient. Thus, in order to determine whether the IPG 10 is capable of delivering effective therapy, and, if so, the stimulation parameters that define such effective therapy, the patient's response to different stimulation parameters is typically evaluated during a trial stimulation phase prior to the permanent implantation of the IPG 10.

As shown in FIG. 3, during the trial stimulation phase, the distal ends of the leads 18 are implanted within the epidural space 302 along the spinal column 304. Implantation of the leads 18 is a relatively simple procedure in which the patient is usually under only light sedation. A local anesthetic is typically administered at the lead insertion site (e.g, in the lower back region), and a needle (e.g., a 14 or 16 gauge needle) is inserted to create a percutaneous opening 306 through the skin 5. The needle is advanced into the epidural space to the desired lead location under fluoroscopic guidance. The lead 18 is then inserted through the needle on a stylet, which acts to stiffen the lead 18 such that it can be maneuvered to the desired location. When the lead is in the desired position (as verified by fluoroscopy), the stylet is withdrawn and the process is repeated for any additional leads 18.

During the trial stimulation phase, the proximal ends of the leads 18 including the electrode terminals 20 are ultimately coupled to an external trial stimulator (ETS) 70, which, as its name implies, is external to (i.e., not implanted in) the patient. An external cable box assembly 340 is used to facilitate the connection between the leads 18 and the ETS 70. Each external cable box assembly 340 includes an external cable box 342 (which has a receptacle similar to connector block 22 for receiving the lead), a trial stimulation cable 344, and a male connector 346, which is plugged into a port 72 of the ETS 70. Once connected to the leads 18, the ETS 70 can then be affixed to the patient in a convenient fashion for the duration of the trial stimulation phase, such as by placing the ETS 70 into a belt worn by the patient (not shown).

The ETS 70 essentially mimics operation of the IPG 10 to provide stimulation to the implanted electrodes 16. This allows the effectiveness of stimulation therapy to be verified for the patient, such as whether therapy has alleviated the patient's symptoms (e.g., pain). Trial stimulation using the ETS 70 further allows for the determination of a particular promising stimulation program for the patient to use once the IPG 10 is later implanted into the patient. Although not shown, the ETS 70 typically contains a battery within its housing along with stimulation and communication circuitry.

The stimulation program executed by the ETS 70 can be provided or adjusted via a wired or wireless link 92 (wireless shown) from a clinician programmer 90. As shown, the clinician programmer 90 comprises a computer-type device, and may communicate wirelessly via link 92 using a communication head or wand 94 wired to the computer. Communication on link 92 may comprise magnetic inductive or short-range RF telemetry schemes as already described, and in this regard the ETS 70 and the clinician's programmer 90 and/or communication head 94 may include antennas compliant with the telemetry means chosen. Clinician programmer 90 may be as described in U.S. Patent Publication No. 2015/0360038. Note that the external controller 40 (FIG. 2) may also communicate with the ETS 70 to allow the patient means for providing or adjusting the ETS 70's stimulation program.

At the end of the trial stimulation phase, the leads 18 (or at least lead extenders that include a portion external to the body) are typically explanted and the relatively small percutaneous openings 306 are closed in a further, yet simple, surgical procedure. If trial stimulation proved ineffective for the patient, no further procedures are performed.

By contrast, if stimulation therapy proved effective, IPG 10 can be permanently implanted in the patient, which is often performed in a subsequent procedure after the trial leads 18 (or lead extenders) are explanted. (“Permanent” in this context generally refers to the useful life of the IPG 10). Permanent implantation involves creating a surgical pocket (e.g., in the buttocks) in which the IPG 10 is positioned, implanting permanent leads 18 using the same technique as described above, subdermally tunneling the proximal ends of the leads 18, including electrode terminals 20, to the pocket, and coupling the leads 18 to the connector blocks 22 in the IPG's header 24. The result is a fully-implanted stimulation therapy solution. The IPG 10 can be programmed with the stimulation parameters that were found to be effective during the trial stimulation phase. Subsequently, the stimulation parameters can be modified wirelessly using either the external controller 40 or the clinician programmer 90.

While this trial stimulation approach can be effective, the inventors have recognized certain drawbacks. A first is that because the leads 18 extend through percutaneous openings 306 during the trial stimulation phase, there is some risk of infection. While proper bandaging and antibiotics can help mitigate this risk, it is not prudent to continue with the trial stimulation phase for an extended period of time. Therefore, the duration of the trial period is typically limited to several days (e.g., 10-14 days), a substantial portion of which time the patient is recovering from the lead implantation procedure. As a result, there is not much time during which the patient can evaluate the effectiveness of various stimulation parameters under “normal” circumstances. In other words, even though it may be desirable in some cases to extend the trial stimulation phase, the need to close the openings 306 may cut the experimental period short, thus forcing a premature decision whether to proceed with implantation of the IPG 10.

A further drawback is that the trial stimulation phase requires two procedures within a short time period. The leads 18 are implanted and then, several days later, the patient undergoes an additional procedure to explant the leads, which can be difficult on the patient.

This has caused the inventors to consider solutions that can either extend the trial stimulation period or even eliminate the requirement of a multi-step implantation procedure.

### BRIEF DESCRIPTION OF THE DRAWINGS

FIG. 1 shows an implantable pulse generator (IPG), in accordance with the prior art.

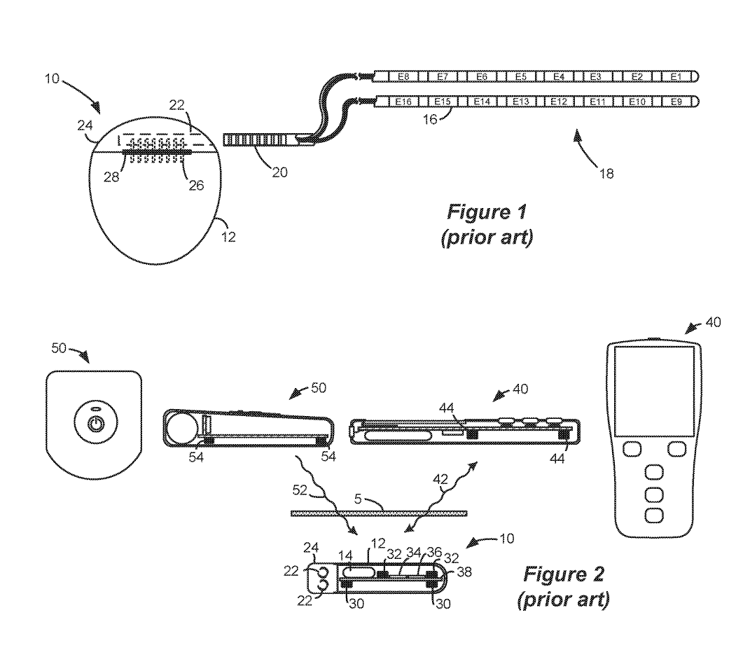

FIG. 2 shows a cross section of the IPG of FIG. 1 as implanted in a patient, as well as external devices that support the IPG, including an external charger and external controller, in accordance with the prior art.

FIG. 3 shows use of trial stimulation preceding implantation of the IPG, including implanted leads/electrodes communicating with an External Trial Stimulator (ETS), in accordance with the prior art.

FIG. 4 shows a powering patch and a convertible stimulator that can be powered continuously by such a powering patch, in accordance with an example of the invention.

FIG. 5 shows circuitry useable in the powering patch and in the convertible stimulator, in accordance with an example of the invention.

FIG. 6 shows different configurations of an electronics module portion of the convertible stimulator, in accordance with various examples of the invention.

FIG. 7 shows a power supply module that can be used in conjunction with the convertible stimulator, in accordance with an example of the invention.

FIGS. 8A-8C show different configurations of a power supply module that can be used in conjunction with the convertible stimulator, in accordance with various examples of the invention.

FIG. 9 shows circuitry useable in the convertible stimulator and the power supply module along with circuitry of supporting external devices, in accordance with an example of the invention.

### DETAILED DESCRIPTION

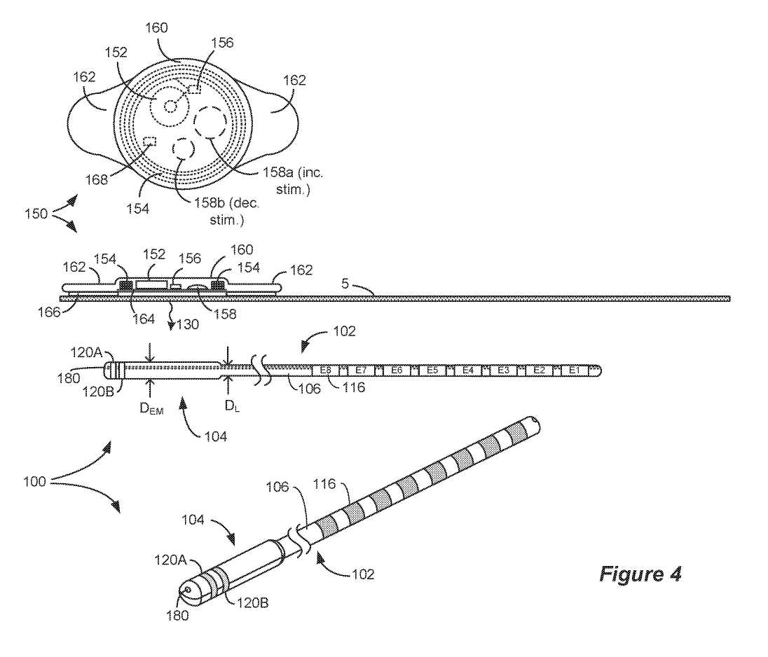

The inventors realize that traditional external trial stimulation techniques as described earlier (FIG. 3) are driven at least in part by the size of the IPG 10. Even though manufacturers labor to make IPGs such as 10 as small as possible (e.g., between 10-40 cm³ in volume at the current time), such IPGs are still significant in size, particularly because the battery 14 (whether rechargeable or primary) is relatively large. It is therefore generally desired by patients and clinicians alike that the IPG 10 only be implanted once stimulation therapy effectiveness has been verified during the ETS trial period. But as mentioned, due to the limited time that the percutaneous openings 306 can prudently remain, trial stimulation enables evaluation over a relatively short time period (e.g., 10-14 days).

Accordingly, the inventors disclose a convertible stimulator system that allows for trial stimulation to occur in a fully implanted solution (i.e., a solution that does not require leads to pass outside of the body through openings such as 306) for an essentially unlimited duration followed by “conversion” of the convertible stimulator to a more traditional system through the implantation and connection of a separate power supply module, if desired. The convertible stimulator includes a lead portion and an electronics module in an integrated package, and it is initially completely implanted without the separate power supply module. As will be described below, the electronics module preferably has a diameter that is similar to that of the lead portion such that the entire convertible stimulator can be easily injected and/or subdermally tunneled to facilitate implantation of the convertible stimulator without any additional risk or inconvenience as compared to the lead implantation procedure described above.

In order to meet these size restrictions, the convertible stimulator preferably does not include an internal battery, although it may include a very small capacity battery or capacitor acting as an internal power source to provide power for a limited duration. The convertible stimulator is instead provided continuous power from a field produced by an external charger device, which may take the form of a powering patch, prior to the implantation and connection of the separate power supply module. A coil or other antenna arrangement in the convertible stimulator picks up and rectifies this field to provide power to stimulating electronics in the convertible stimulator, and also to recharge the small battery or capacitor if present.

Should stimulation therapy as provided by the convertible stimulator prove ineffective, the convertible stimulator may be explanted at a convenient later time not dictated by considerations of infection risk due to percutaneous openings, which again are not present in the disclosed technique. Conversely, should stimulation therapy prove effective, the convertible stimulator can continue to be used by the patient for stimulation during an extended trial period or even beyond, although such stimulation will require use of the continuous external charger. Should it eventually be decided that stimulation therapy is effective enough to warrant conversion to a more traditional system (i.e., a system that does not require the use of a continuous external power supply), the separate power supply module can be implanted and coupled to the convertible stimulator at a convenient time for the patient and clinician. Because the convertible stimulator is designed to accommodate such a conversion and the power supply module can be small (especially if it employs a rechargeable power supply), the “conversion” procedure simply involves the creation of a pocket to accommodate the power supply module and the connection of the power supply to the electronics module (which is initially implanted at a site that can accommodate subsequent conversion, e.g., in the buttocks). Eventual conversion of the convertible stimulator through the connection of the implanted power supply (while not strictly required) can convenience the patient, who will no longer need to ensure that power is continuously applied to the convertible stimulator.

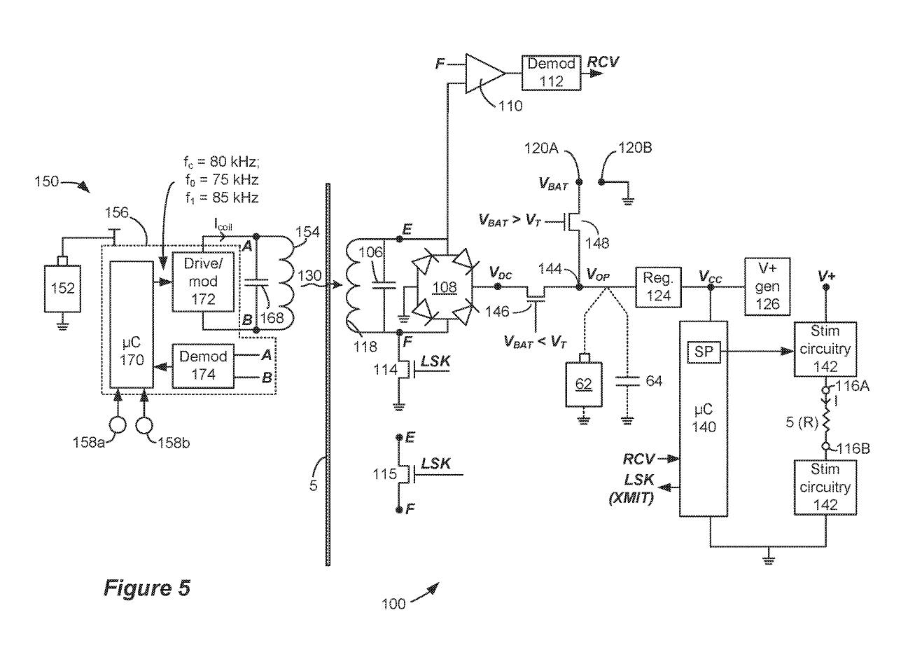

An example of a convertible stimulator 100 as described above and as implanted in a patient's tissue 5 is shown in FIG. 4. As shown, the convertible stimulator 100 includes a lead portion 102 and an electronics module portion 104. The lead portion 102 is similar to a traditional electrode lead (e.g., lead 18) and has a lead body 106 along which a number of electrodes 116 are positioned. While eight ring-type electrodes are shown, other numbers and types of electrodes 116 can be used. The lead body 106 is formed of a biocompatible, non-conducting material such as, for example, a polymeric material like silicone, polyurethane, polyurea, polyurethane-urea, polyethylene, or the like. The electrodes 116 may be formed from a metal, alloy, conductive oxide, or any other suitable conductive biocompatible material such as platinum, platinum iridium alloy, iridium, titanium, tungsten, palladium, palladium rhodium, or the like.

The electronics module 104 of the convertible stimulator 100 has a generally cylindrical shape with rounded edges to ensure patient comfort. The electronics module 104 is formed of a biocompatible material such as titanium, a ceramic material, or an epoxy, and, in the example shown, has a slightly larger diameter (D_{EM}) than that of the lead portion 102 (D_{L}). The difference in diameter in the illustrated example is not required and, in other embodiments, the electronics module 104 may have the same diameter as the lead portion 102. In any event, the largest diameter of any portion of the convertible stimulator 100 is preferably small enough to enable it to pass within a standard gauge (e.g., 14 gauge) needle or at least to be easily subdermally tunneled. For example, the electronics module 104 may have a diameter of 1.6 mm or less and the lead portion 106 may have a diameter of 1.5 mm or less. The length of the electronics module 104 may be approximately 1-2 inches or less and is generally dictated by the size of the electrical components that are housed within the electronics module 104, which components are described below. The short length of the electronics module improves MM compatibility. Although the electronics module 104 has been described as having a cylindrical shape, in an alternative embodiment, the electronics module 104 may have an oblong (e.g., oval) cross section.

One or more electrical contacts 120 are positioned on the exterior portion of the electronics module 104. While two ring-type contacts are shown, different numbers and types of contacts might also be used. As described in detail below, the contacts 120 are coupled to circuitry within the electronics module 104 and provide a connection point to establish electrical power and communication between such circuitry and electrical components within the separate power supply module if and when the convertible stimulator 100 is connected to such a separate power supply module. It will be understood that if the housing of the electronics module 104 is formed of a conductive material, the contacts 120 are isolated from the housing by an insulating material.

An optional stylet channel 180 extends through the convertible stimulator 100 from the proximal end of the electronics module 104 through the distal end of the lead portion 102. This channel enables the insertion of a stylet to stiffen the lead portion 102 of the convertible stimulator 100 during implantation. A typical implantation of the convertible stimulator 100 involves implantation of the lead portion 102 through a standard gauge needle (with a stylet inserted and in a manner that mirrors the lead implantation procedure described in the background section above) followed by the subdermal tunneling of the electronics module 104 to a suitable location for the possible subsequent implantation of the power supply module (e.g., the buttocks).

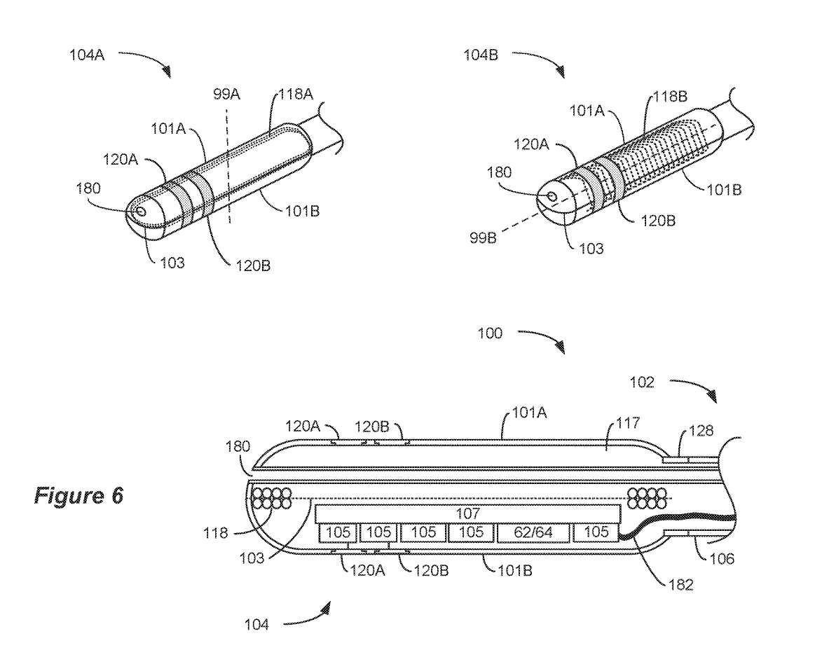

Because the convertible stimulator 100 may lack an internal power source altogether, or may include only a small rechargeable battery or capacitor, an external powering device such as a powering patch 150 is used to provide continuous power to the convertible stimulator 100 prior to the connection of an implanted power supply module. As shown in FIG. 4 and in the circuit diagram of FIG. 5, the patch 150 includes a battery 152 (preferably of a flat configuration, such as a coin-shaped battery), a primary coil 154 for producing a magnetic field 130, a capacitor 168 for tuning the frequency of the magnetic field in conjunction with an inductance of the coil 154, and various circuitry 156 shown in further detail in FIG. 5. Although the coil 154 and capacitor 168 are shown in FIG. 5 connected in parallel to create a resonant tank circuit, they may also be connected in series as is well known.

In one example, the magnetic field 130 produced by the patch 150 can comprise 80 kHz (f_{c}). The magnetic field 130 is in turn received at a secondary coil 118 (FIG. 5) in the convertible stimulator 100. A capacitor 106 (FIG. 5) in the convertible stimulator 100 is used to set the resonant frequency of the IPG's tank circuit (118/106) to that of the magnetic field 130 for efficient reception. The received magnetic field 130 is rectified (108) and used to produce a DC voltage in the convertible stimulator 100, V_{DC}.

When the transistor 146 is in the closed position and the transistor 148 is in the open position, which occurs when the voltage at contact 120A, V_{BAT}, is less than a predetermined threshold voltage, V_{T}, V_{DC} is passed to node 144 to provide the operating voltage, V_{OP}, for the convertible stimulator 100. By contrast, when V_{BAT} is greater than V_{T}, the transistor 146 is in the open position and the transistor 148 is in the closed position such that V_{BAT} is passed to node 144 to provide the operating voltage, V_{OP}, for the convertible stimulator 100. The threshold voltage, V_{T}, can be selected to be a value just above the fully depleted voltage value of a power source (described below) within the power supply module and can be programmed into the microcontroller 140, which generates the control signals that set the states of the transistors 146 and 148. In this manner, the operating power for the convertible stimulator 100 is provided from the received field 130 when the power supply module is not connected to the convertible stimulator 100 and is provided from the power supply module when it is connected (and is not fully depleted). Although the described embodiment defaults to the use of V_{BAT} when it exceeds a threshold voltage, an alternate embodiment may default to the use of V_{DC} when it exceeds the threshold voltage. In such an embodiment, the operating voltage may be derived from the field 130 generated by an external device even when the power supply module has been connected, which may occur, for example, each time a rechargeable battery in the power supply module is charged.

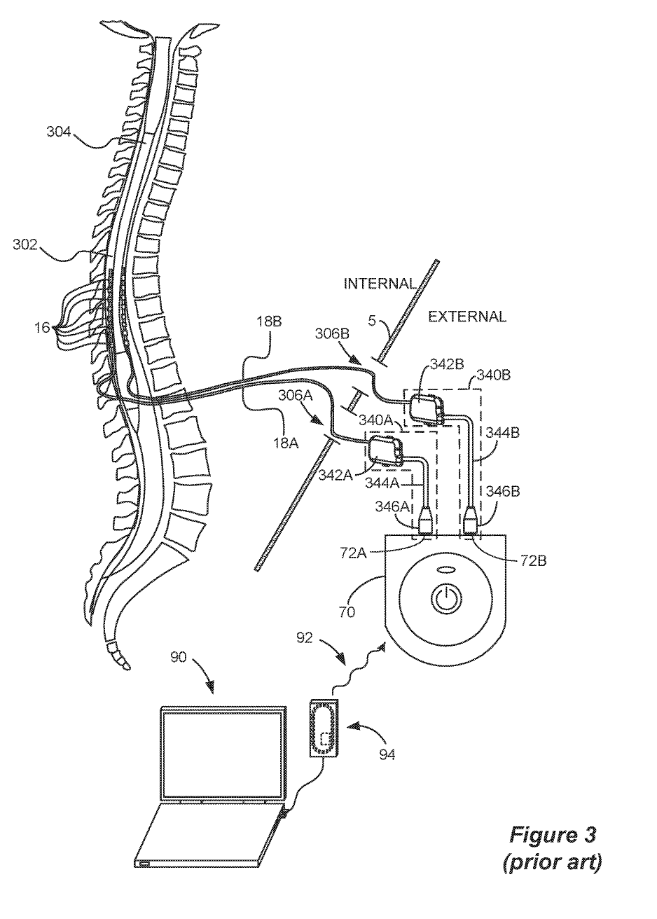

V_{OP} can in one embodiment represent the sole power source for the convertible stimulator 100's circuitry, and therefore require the patch 150 (or alternate external power source) to be present and providing a magnetic field 130 or the power supply module to be connected via terminals 120 for any aspect of the convertible stimulator 100 to operate. Alternatively, the convertible stimulator 100 can as shown in dotted lines in FIG. 5 include a small capacity power source, such as rechargeable battery 62, or a capacitor 64 which can be charged to provide power for a small time (e.g., a few minutes). Having a small power source can be useful, especially when no power supply module is connected, to attend to various housekeeping functions in the convertible stimulator 100, such as data storage, or possibly to allow communication with an external controller (e.g., 40 or 90, FIG. 2 or 3), which may for example be used to program the convertible stimulator 100 or set or adjust its stimulation program. If necessary, a regulator 124 can be included to smooth the operating voltage to a reliable voltage, V_{CC}, which can be used to power circuitry in the convertible stimulator 100, such as the microcontroller 140. Further, V_{OP} (or V_{CC}) can be boosted to a higher voltage by a generator 126, such as by boost circuitry that is used to produce a compliance voltage, V+, that powers the stimulation circuitry 142. Use of boost circuitry to generate V+ is discussed in U.S. Pat. No. 7,444,181, for example. Stimulation circuitry 142 is coupled to the electrodes 116 and provides electrical stimulation to the patient's tissue 5 via the electrodes 116 in accordance with a stimulation program.

Preferably, the patch 150 can alter the strength of the magnetic field 130 it produces using telemetered feedback from the convertible stimulator 100. Thus, circuitry 156 includes a demodulator 174 for decoding data wirelessly received from the convertible stimulator 100; control circuitry (such as a microcontroller) 170 for interpreting such data; and drive and modulation circuitry 172. Drive and modulation circuitry 172 can set the strength of the AC current (I_{coil}) that will flow through the patch's coil 154 and hence the strength of the magnetic field 130 it produces. Data regarding how to set I_{coil} can come from telemetry circuitry in the convertible stimulator 100, which may transmit data to the patch 150 via Load Shift Keying (LSK) for example. As is known, LSK involves modulating the impedance of the coil 118 in the convertible stimulator 100 with data to be transmitted to the patch 150, which causes decodable perturbations in the magnetic field 130 the patch 150 produces. Convertible stimulator 100 thus includes LSK circuitry for this purpose, represented as a transistor 116 capable of selectively shorting both ends of the coil 118 together in accordance with the data to be transmitted. LSK circuitry may also selectively short both ends of the coil 118 to ground, as represented by transistor 114. Telemetry of data from an implantable medical device to an external charger via LSK is discussed further in U.S. Patent Application Publication 2015/0080982. While magnetic field 130 adjustments are desirable, for example to ensure that V_{DC} is set to a proper level, it isn't strictly necessary that all embodiments of patch 150 have such capability, and instead continuous magnetic field 130 can be non-adjustable.

As discussed earlier, the patch 150 preferably also includes the ability to transmit data to the convertible stimulator 100 via drive and modulation circuitry 172. For example, at times when the patch 150 is used to change the stimulation program running in the convertible stimulator 100 (more on this below), data can be modulated on the magnetic field 130 using Frequency Shift Keying (FSK). In one example, the magnetic field 130 may be tuned to a center frequency (f_{c}) of 80 kHz when not modulated with data and merely providing power, but may vary its frequency (e.g., f₀=75 kHz; f₁=85 kHz) when sending ‘0’ and ‘1’ data bits. Alternatively, data may be modulated on magnetic field 130 by various forms of amplitude or phase modulation. The convertible stimulator 100 may receive this data at an amplifier 110 connected to the receiving coil 118, which outputs the amplified data to demodulation circuitry 112, which in turn reports this data in digital form to a microcontroller 140 in the convertible stimulator 100. Such received data can include a stimulation program as discussed above, which informs stimulation circuitry 142 in the convertible stimulator 100 which electrodes 116 to stimulate and how to so stimulate them (e.g., frequency amplitude, duration, etc.). Stimulation circuitry 142 may be as described in U.S. Pat. Nos. 8,606,362 and 8,620,436 for example. While communications between an external device and the convertible stimulator 100 have been described in the context of communications via magnetic induction using the coil 118, the convertible stimulator 100 may also include a separate communications antenna that enables communications via other known short-range RF telemetry schemes (e.g., Bluetooth, WiFi, Zigbee, MICS, etc.).

The patch 150 is preferably light weight and disposable, and may generally resemble an adhesive bandage in structure. It is contemplated that the magnetic field 130 will be continuously produced until the battery 152 in the patch 150 is depleted, at which time a new patch 150 would need to be affixed to the patient. Alternatively, the battery 152 may be replaceable in the patch 150, thus allowing the patch to be re-used.

Referring again to FIG. 4, the housing 160 of the patch 150 may be made for example of a soft plastic material between which the coil 154 and other electronic components are sandwiched. If necessary, wings 162 outside of the area of the electronics may be included to promote affixation to the patient's skin above the implanted location of the convertible stimulator 100, and an adhesive 166 may be positioned under the wings 162. Alternatively, wings 162 may not be needed, and adhesive 166 can instead be placed underneath the electronics of the patch 150. While use of an adhesive 166 is preferred to affix the patch 150 at the location of the convertible stimulator 100, this is not strictly required and other means of positioning the patch 150 can be used as well. Although not shown, the patch 150 electronics can be supported within the housing 160 by a substrate, preferably a flexible substrate such as formed of Kapton for example. Further details concerning a patch such as 150 can be found in U.S. Patent Publication No. 2016/0367822.

Due to its preferably simple construction, the patch 150 may contain no user interface elements. Alternatively, the patch may include simple means for adjusting the stimulation therapy being provided by the convertible stimulator 100. For example, the electronics of the patch 150 may include depressible bubble contacts 158a and 158b that are used to increase and decrease the amplitude of stimulation being provided by the stimulation program (SP) the convertible stimulator 100 is currently running. Notice that bubble contact 158a may be larger than bubble contact 158b, thus providing the patient easy means to feel which of the two contacts is to be used for increasing and decreasing stimulation. Alternatively, other devices may be used to provide power and also the data necessary to adjust stimulation therapy, such as the external controller 40 (FIG. 2) or clinician programmer 90 (FIG. 3) described earlier, thus leaving patch 150 to only provide operating power for the convertible stimulator 100. Such other means of communicating data to and from the convertible stimulator 100 may also occur at different frequencies than the continuously-provided magnetic field 130, thus allowing data communications to take place while the magnetic field 130 is present.

While a light weight patch 150 is preferred that can be fixed in position on the patient's skin relative to the convertible stimulator 100, convertible stimulator 100 can alternatively be powered by other external charging devices. For example, the convertible stimulator 100 may be powered by more traditional external charging devices, such as the external charger 50 described earlier (FIG. 2), even if the use of such bulkier devices, and/or devices which may move relative to convertible stimulator 100, would be less convenient for the patient.

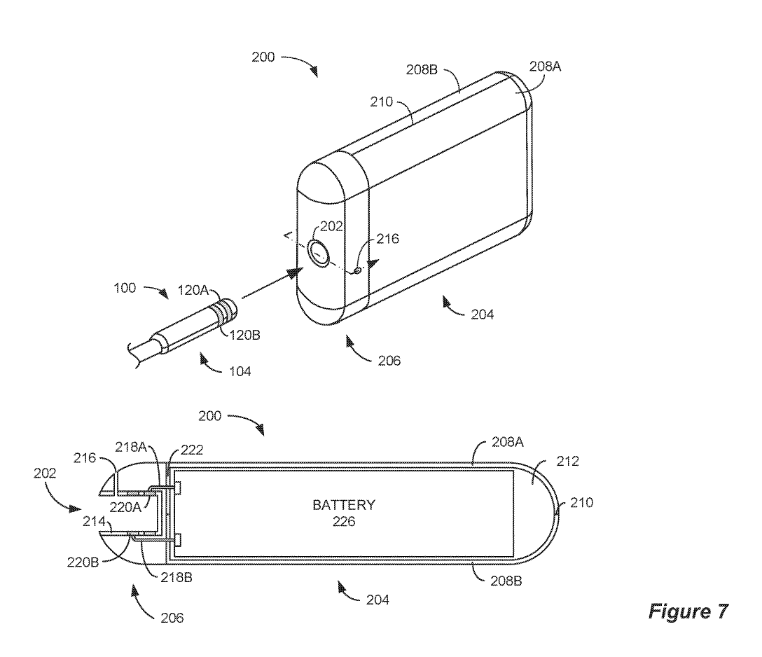

FIG. 6 shows further details of the construction of the convertible stimulator 100 shown earlier in FIG. 4. The top portion of FIG. 6 shows examples of electronics modules 104A and 104B having different coil arrangements. Electronics module 104A's coil 118A is wound around an axis 99A that is perpendicular to the plane of the patch 150's coil 154, while electronic module 104B's coil 118B is wound around an axis 99B that is parallel to the plane of the patch 150's coil 154. Due to its larger area and orientation, the magnetic flux through the coil 118A is greater than that through the coil 118B; however, the orientation of the coil 118B allows a greater number of turns without interfering with the components that are positioned within the electronics module 104. As a result, the optimal orientation of the coil 118 must be determined on the basis of the specific properties of the convertible stimulator 100 (e.g., the size of the electronics module 104 and positioning of electrical components therein) as well as the properties of the continuous charger (e.g., the strength of the field 130, etc.). While the examples shown utilize coils 118 to provide operating power for the convertible stimulator from a received magnetic field, other embodiments may employ different types of antennas for extracting energy from an electrical field generated by the external power supply.

Each of the example electronics modules 104A and 104B includes a housing 101 that is formed of top and bottom “clamshell” portions 101A and 101B that are joined (e.g., welded or brazed) at a seam 103, although other methods of construction, including molding the convertible stimulator 100 as a single unit, are also possible. As shown, the coils 118A and 118B are positioned within the housing 101. It will be understood that if the housing portions 101A and 101B are formed of a conductive material, such as titanium, then they will attenuate the magnetic field 130 to some degree, and as such, the housing portions 101A and 101B may instead be formed of non-conductive materials, such as ceramic or epoxy, as described above.

The bottom portion of FIG. 6 shows a cross-sectional view of the electronics module 104 and its connection to the lead portion 102 (note that the coil 118 is arranged as in electronics module 104A). The lead portion 102 is received within an opening in the distal end of the electronics module 104. More specifically, a connector 128, such as a ceramic or metal connector that is formed as an integral piece of the lead body 106, is connected to the housing portions 101A and 101B, such as by welding or brazing, to form the convertible stimulator 100 as an integrated unit (i.e., the lead portion 102 is not detachable from the electronics module 104). The stylet channel 180 and the conductor wires 182 that are connected to the electrodes 116, which channel 180 and wires 182 are also formed as part of the lead body 106, extend into the interior portion of the electronics module 104. The stylet channel 180, which may be formed as a small diameter piece of tubing that is molded within the lead body 106, is fitted into and connected to a small hole in the proximal end of the housing portion 101A and is cut so that it is flush with the exterior of the housing portion 101A. The various components (i.e., the lead portion 102 and the stylet channel 180) are connected to the electronics module 104 in a manner that creates a cavity 117 (which may be hermetic) within the interior of the electronics module 104.

Although the electronics module 104 and the lead portion 102 have been described as separate components that are fixed together to form an integrated unit, the different portions may also be initially formed as single component. For example, the various mechanical and electrical components may be positioned within a mold cavity and overmolded to create the convertible stimulator 100 as a single integrated unit. In such an embodiment, the electronics module 104 would not include a cavity 117, but rather the components would be encapsulated within a mold material. Regardless of the way in which the convertible stimulator 100 is constructed, because it is an integrated unit, the lead portion 102 may be provided in several different length options to accommodate the patient-specific distance between the desired locations of the electrodes 116 and the electronics module 104. Moreover, although the illustrated device includes electrodes arranged on a percutaneous lead portion 102, other electrode arrangements, such as a two dimensional arrangement of electrodes on a paddle style lead, may also be used.

Cavity 117 contains a printed circuit board (PCB) 107, which includes electronic components 105 that make up the circuitry described in FIG. 5. Additionally, PCB 107 may include a small optional rechargeable battery 62 or storage capacitor 64 as mentioned previously. The contacts 120A and 120B are electrically coupled to electrical components 105 on the PCB 107 to accomplish the functionality described with respect to FIG. 5. The cavity 117 also houses the coil 118, which is oriented as shown in FIG. 5 and described above.

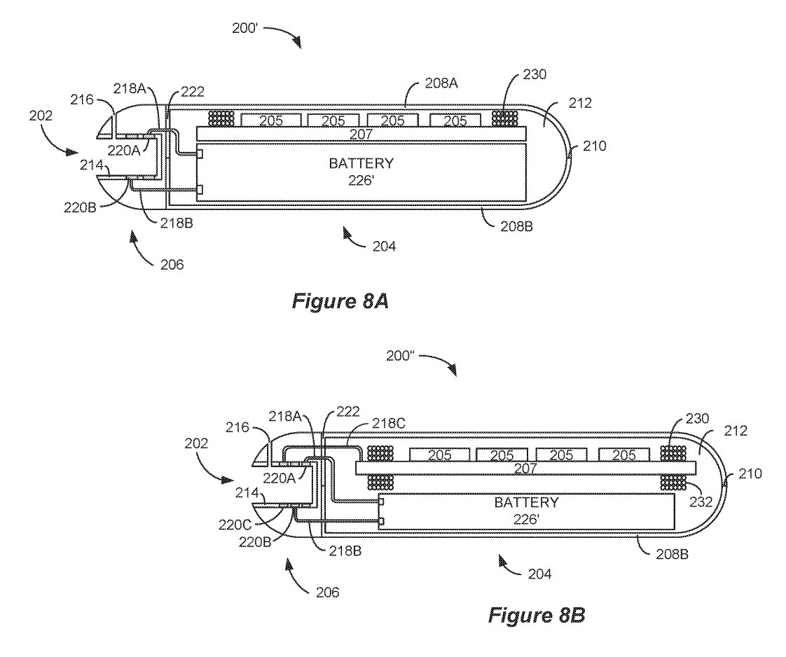

FIG. 7 shows an example of a power supply module 200 that can optionally be connected to the convertible stimulator 100 after a successful trial period to provide a more traditional SCS system (i.e., a system that does not depend on continuous external power). The power supply module 200, which is generally rectangular-shaped with rounded edges and which may have a volume of approximately 20 cm³, can be implanted into a surgically-created pocket near the proximal end of the convertible stimulator 100. The power supply module 200 includes a housing 204 and a header 206. The housing 204 is formed from separate “clamshell” housing portions 208A and 208B, which may be constructed from a biocompatible material such as a ceramic or titanium. The housing portions 208A and 208B are joined, such as by brazing or welding along a seam 210, to create the housing 204, which encloses a hermetic cavity 212, although other methods of constructing the housing 204 may also be employed. The header 206, which can be formed from an epoxy for example, continues the general shape of the housing 204 and has an opening 202 in which a connector block 214 is positioned. The connector block 214 includes contacts 220, which are configured to align with the contacts 120 on the outer portion of the electronics module 104 when the electronics module 104 is inserted into the connector block 202 as shown. A set screw channel 216 extends from the exterior of the header 206 into the connector block 214 and is configured to receive a set screw that can be advanced to contact the electronics module 104 and maintain the electronic module 104's position within the connector block 214. Although the power supply module 200 includes a set screw, other mechanisms for maintaining the position of the electronics module 104 could also be employed.

As illustrated in the cross-sectional view in the bottom portion of FIG. 7, a battery 226 (which, in the case of power supply module 200 is a primary battery) occupies substantially all of the volume of the hermetic cavity 212. The battery 226's terminals are coupled to the contacts 220 via feedthrough pins 218 that extend through a hermetic feedthrough 222 and into the header 206. In this way, the battery 226 provides the operating voltage, V_{OP}, at the node 144 (FIG. 5) to power the convertible stimulator 100. A primary battery such as the type positioned in the power supply module 200 may have a capacity of 7 AHr and may be expected to provide a voltage that is sufficient to power the convertible stimulator 100 for a period of 3-7 years. Therefore, following a successful trial period in which the convertible stimulator 100 is operated independently, the power supply module 200 can be connected and implanted to provide more traditional SCS therapy for an extended duration. Moreover, should the battery 226 be depleted at some point in the future, the convertible stimulator 100 can again be operated essentially indefinitely by an external power supply such as the patch 150, for example, until a future treatment decision (e.g., replacing the power supply module 200, replacing the convertible stimulator 100 and power supply module 200 with a different device, etc.) can be made.

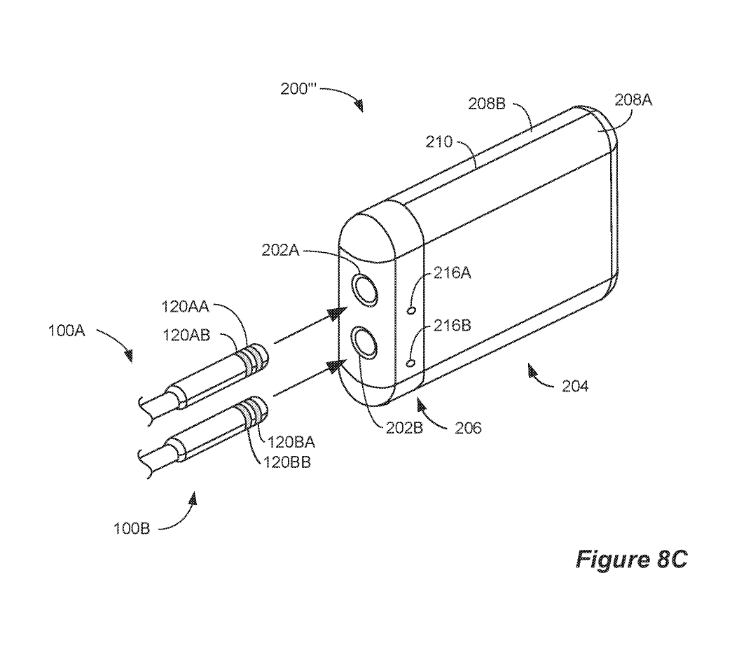

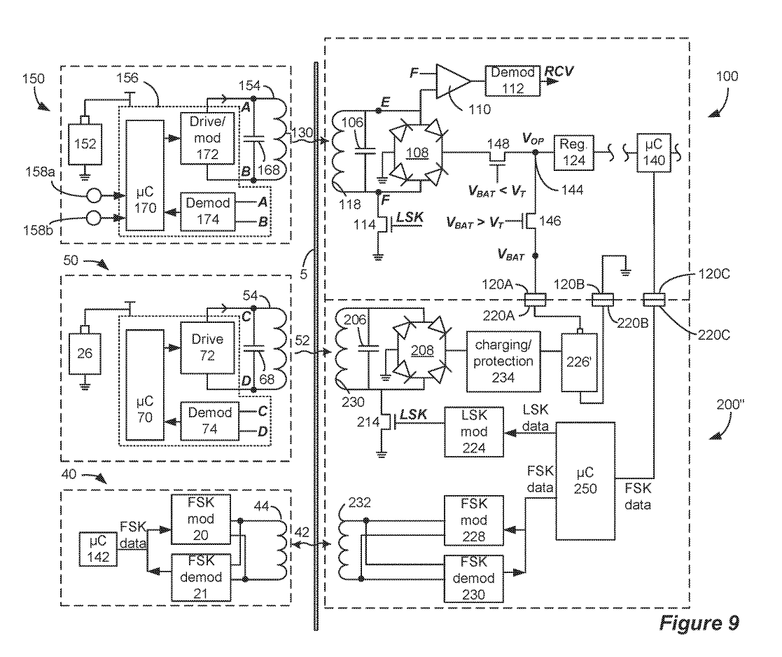

FIG. 8A shows a cross-sectional view of a power supply module 200′ that is similar in construction to power supply module 200 but which includes a rechargeable battery 226′, which powers the convertible stimulator 100 as well as circuitry within the power supply module 200′. The use of a rechargeable battery may allow the power supply module 200′ to be designed with a smaller volume than the power supply module 200. The cavity 212 of the power supply module 200′ houses a charging coil 230, which receives energy from a magnetic field induced by an external charger such as the charger 50, for example, and converts the energy from the magnetic field into a current that is utilized to recharge the battery 226′. The electrical circuitry that is utilized to recharge the battery 226′, which is illustrated in FIG. 9, is implemented by electrical components 205 that are positioned on a circuit board 207, which are also located within the cavity 212.

FIG. 8B shows a cross-sectional view of a power supply module 200″ that is similar in construction to the power supply modules 200 and 200′ but which includes both a rechargeable battery 226′ and telemetry circuitry. The telemetry circuitry includes a telemetry coil 232 that is configured to receive data telemetered from an external device such as the external controller 40 (FIG. 2) or clinician programmer 90 (FIG. 3). Because it is significantly larger than the electronics module 104, the power supply module 200″ may be capable of housing a telemetry coil 232 that is substantially larger and/or better oriented with an external coil than the coil 118, thus improving the efficiency with which data telemetry can be conducted. The electrical circuitry that is utilized to recharge the battery 226′ and to conduct data telemetry, which is illustrated in FIG. 9, is implemented by electrical components 205 that are positioned on a circuit board 207, which are also located within the cavity 212. The use of power supply module 200″ for data telemetry requires a data connection between the electronics module 104 and the power supply module 200″, which is accomplished through the connection of an additional contact 220C in the connector block 214 to electrical components 205 on the PCB 207 via an additional feedthrough pin 218C. While a single additional contact 220C and feedthrough pin 218C are shown, it will be understood that additional functionality may require additional contacts 220 and feedthrough pins 218 and that additional contacts 220 correspond to additional contacts 120 on the exterior of the electronics module. In an alternate embodiment, the telemetry coil 232, which is shown as being positioned within the cavity 212, may instead be positioned in the header 206. Moreover, although a telemetry coil 232 for communications via magnetic induction is shown, other known short-range RF telemetry schemes (e.g., Bluetooth, WiFi, Zigbee, MICS, etc.) can also be used and therefore the telemetry coil 232 may be replaced by a different type of antenna that is appropriate for the chosen telemetry scheme.

FIG. 8C shows a perspective view of yet another power supply module 200′″, which is constructed similarly to the previously-described power supply modules but which includes two openings 202A and 202B to accommodate two separate convertible stimulators 100A and 100B. While the convertible stimulators 100A and 100B are depicted with two contacts 120 each, it will be understood that they may include additional contacts to accommodate additional functionality of the power supply module (e.g., telemetry circuitry). By accommodating two separate convertible stimulators 100, the power supply module 200′″ enables the convertible stimulators 100A and 100B to mimic a more traditional SCS system with both right and left leads, for example. In one embodiment, the power supply module 200′″ may include circuitry to synchronize the convertible stimulators 100A and 100B such that they may cooperate in the delivery of stimulation therapy. In this regard, the combined system (i.e., the power supply module 200′″ and the convertible stimulators 100A and 100B) may have improved stimulation capabilities as compared to the convertible stimulators 100A and 100B acting independently.

While the example power supply modules include openings 202 that accommodate insertion of an electronics module 104 in a direction along the length of the power supply module, the openings 202 may alternatively be positioned to accommodate insertion of an electronics module in a direction along the width of the power supply module. Likewise, while the described power supply modules have included batteries 226, 226′ as the power source, it will be understood that other types of power sources, such as a supercapacitor, for example, may be used. While several example power supply modules have been described, it will be understood that different designs and functionality may be implemented. For example, the features of the described power supply modules may be combined and additional features may be added.

The convertible stimulator 100 may in one embodiment be designed to be compatible with multiple power supply modules (such as power supply modules 200, 200′, 200″ and 200′″) to enable a patient to select the appropriate power supply module for their needs. For example, a patient that determines during an extended trial period (i.e., in which the convertible stimulator 100 acts independently) that the type of therapy they find effective is energy intensive, may be best served by a power supply module with a rechargeable battery, such as power supply modules 200′ or 200″. Conversely, a patient that determines during an extended trial period that the type of therapy they find effective is not energy intensive, may prefer a power supply module with a non-rechargeable battery, such as power supply module 200.

FIG. 9 illustrates the combined electrical circuitry of the convertible stimulator 100 connected to the power supply module 200′, which, as described above, includes a rechargeable battery 226′ and data telemetry capabilities, along with the circuitry of corresponding external devices such as patch 150, external controller 40, and external charger 50. The electrical circuitry of the patch 150 and the convertible stimulator 100 are as described above with respect to FIG. 5, although some components have been removed for purposes of clarity. The insertion of the electronics module 104 of the convertible stimulator 100 within the opening 202 in the power supply module 200′ results in the connection of corresponding contacts 120 and 220. Through the connection of the contacts 120 and 220, the battery 226′ is coupled to the circuitry in the convertible stimulator 100. As described above, if the battery's voltage V_{BAT} exceeds a specified threshold voltage V_{T}, the battery 226′ will supply the operating voltage, V_{OP}, for the convertible stimulator 100 as described above. While a direct connection between the battery 226′ and the contacts 220 is shown, an inverter may be interposed to provide an alternating current source to the convertible stimulator 100, which may increase safety and decrease the risk of corrosion at the contacts 120 and 220.

The external charger 50 is used to charge (or recharge) the battery 226′. A battery 26 in the external charger 50 provides operational power for the charger 50 and energy for the production of a magnetic charging field 52. Specifically, and as described above with respect to FIG. 2, the external charger 50 contains a coil 54 that is energized via drive circuit 72 with a non-modulated AC current to create the magnetic charging field 52. The magnetic field 52 induces a current in the charging coil 230 within the power supply module 200′, which current is rectified (208) to DC levels, and used to recharge the battery 226′, perhaps via a charging and battery protection circuit 234 as shown. The frequency of the magnetic charging field (e.g., 80 kHz) may differ from that used for FSK telemetry (nominally 125 kHz) via the coil 232. As mentioned briefly above, the charger 50 can also be employed to provide continuous power to the convertible stimulator 100 directly prior to the implantation of a power supply module or when the battery 226′ is depleted to a level at which its voltage is below the threshold voltage, V_{T}. It should be noted, however, that if the battery 226′ is depleted to such an extent that its voltage is below the threshold voltage, V_{T}, use of the charger 50 will provide continuous power to the convertible stimulator via the coil 118 as well as recharging the battery 226′ via the coil 230 until the battery's voltage exceeds the threshold voltage, V_{T}, at which point the convertible stimulator 100 will switch to operating on battery power.

The power supply module 200″ can also communicate data back to the external charger 50 using Load Shift Keying (LSK) modulation circuitry 224. LSK modulation circuitry 224 receives data to be transmitted back to the external charger 50 from the power supply module's microcontroller 250, and then uses that data to modulate the impedance of the charging coil 230. The coil 230's impedance is modulated via control of transistor 214, which shorts both ends of the coil 230 to ground. Impedance modulation could alternatively be accomplished by shorting both ends of the coil 230 together. The change in impedance is reflected back to coil 54 in the external charger 50, which interprets the reflection at LSK demodulation circuitry 74 to recover the transmitted data. This means of transmitting data from the power supply module 200″ to the external charger 50 is useful to communicate data relevant to charging of the battery 226′, such as the battery level, whether charging is complete and the external charger can cease, and other pertinent charging variables. However, because LSK works on a principle of reflection, such data can only be communicated from the power supply module 200″ to the external charger 50 during periods in which the external charger 50 is active and is producing a magnetic charging field 52.

The external controller 40 is used to send and receive data to/from the power supply module 200″ and, ultimately, the convertible stimulator 100. For example, the external controller 40 can send programming data such as therapy settings to the convertible stimulator 100 to dictate the therapy the convertible stimulator 100 will provide to the patient. Also, the external controller 40 can act as a receiver of data from the convertible stimulator 100, such as various data reporting on the convertible stimulator's status. The external controller 40 is powered by a battery (not shown), but could also be powered by plugging it into a wall outlet, for example.

Wireless data transfer between the power supply module 200″ and the external controller 40 preferably takes place via inductive coupling in generally the same way as described above with respect to the IPG 10. When data is to be sent from the external controller 40 to the power supply module 200″ via FSK link 42, coil 44 is energized with alternating current (AC), which generates a magnetic field, which in turn induces a voltage in the power supply module's telemetry coil 232. The generated magnetic field is FSK modulated (20) in accordance with the data to be transferred. The induced voltage in coil 232 can then be FSK demodulated (230) at the power supply module 200″ back into the telemetered data signals. Data telemetry in the opposite direction via FSK link 42 from the power supply module 200″ to the external controller 40 occurs similarly.

Data that is received from the external controller 40 or that is transmitted to the external controller 40 is communicated between the power supply module's microcontroller 250 and the convertible stimulator's microcontroller 140 over a communications bus that is established through the connection of the contacts 120C and 220C as shown. Connection of the contacts 120C and 220C may cause the convertible stimulator 100 to deactivate its own internal demodulation circuitry 112, which otherwise remains active such that the convertible stimulator 100 can communicate with an external device such as the external controller 40 in the absence of a power supply module that includes data telemetry functionality.

The external controller 40 typically comprises a user interface similar to that used for a portable computer, cell phone, or other hand held electronic device. The user interface typically comprises touchable buttons and a display, which allows the patient or clinician to send therapy programs to the convertible stimulator 100, and to review any relevant status information reported from the convertible stimulator 100.

The disclosed convertible stimulator provides the benefits of a fully implanted IPG that is externally powered during an extended trial period (or permanently, if desired) as well as the benefits of a more traditional internally-powered system through the subsequent connection of a separately-implanted power supply module.

While the invention herein disclosed has been described by means of specific embodiments and applications thereof, numerous modifications and variations could be made thereto by those skilled in the art without departing from the scope of the invention set forth in the claims.

What is claimed is:

1. An implantable stimulator, comprising:
a lead portion having a plurality of electrodes; and
stimulation circuitry electrically coupled to the plurality of electrodes,
wherein the implantable stimulator is configured to receive continuous operating power from an external power supply in a first mode of operation and from a separate implantable power supply module that is connectable to the implantable stimulator in a second mode of operation.

2. The implantable stimulator of claim 1, further comprising one or more external contacts that are connectable to one or more corresponding contacts of the power supply module.

3. The implantable stimulator of claim 1, wherein the lead portion is configured to be implanted through a needle.

4. The implantable stimulator of claim 1, wherein the stimulation circuitry is positioned within an electronics module.

5. The implantable stimulator of claim 4, wherein the electronics module is cylindrical.

6. The implantable stimulator of claim 4, wherein the electronics module and the lead portion are formed as an integrated unit.

7. The implantable stimulator of claim 4, further comprising a stylet channel that extends through the electronics module and the lead portion.

8. The implantable stimulator of claim 1, further comprising a communications connection that is connectable to the power supply module.

9. The implantable stimulator of claim 1, further comprising a microcontroller that is configured to cause the implantable stimulator to switch between the first mode of operation and the second mode of operation based on a voltage at a contact that is connectable to a corresponding contact of the power supply module.

10. The implantable stimulator of claim 1, further comprising a microcontroller that is configured to cause the implantable stimulator to operate in the first mode of operation when a voltage derived from a field generated by the external power supply exceeds a threshold value.

11. An implantable stimulator, comprising:
a lead portion having a plurality of electrodes; and
an electronics module, comprising:
circuitry configured to produce a first voltage from a received magnetic field; and
a contact that is configured to receive a second voltage from a power supply module that is connectable to the implantable stimulator,
wherein the first voltage provides operating power for the implantable stimulator in a first mode of operation and the second voltage provides operating power for the implantable stimulator in a second mode of operation.

12. The implantable stimulator of claim 11, further comprising a microprocessor that is configured to cause the implantable stimulator to switch to the second mode of operation when the second voltage received at the contact exceeds a threshold voltage.

13. The implantable stimulator of claim 11, further comprising a communications antenna.

14. The implantable stimulator of claim 11, wherein the circuitry comprises a coil.

15. The implantable stimulator of claim 14, further comprising circuitry to modulate an impedance of the coil to communicate data to an external device generating the magnetic field.

16. The implantable stimulator of claim 11, further comprising demodulation circuitry to identify data that is transmitted via the magnetic field.

17. The implantable stimulator of claim 11, wherein the lead portion and the electronics module are formed as an integrated unit.

18. The implantable stimulator of claim 11, wherein the lead portion is configured to be implanted through a needle.

19. A system, comprising:
an external power supply; and
a convertible implantable stimulator, comprising:
a lead portion having a plurality of electrodes;
stimulation circuitry configured to provide electrical stimulation to a patient's tissue through one or more of the plurality of electrodes;
a coil configured to receive a magnetic field generated by the external power supply to provide operating power for the convertible implantable stimulator in a first mode of operation; and
one or more contacts that are connectable to corresponding contacts of a separate power supply module, wherein the operating power for the convertible implantable stimulator is provided from a power source in the power supply module in a second mode of operation.

20. The system of claim 19, wherein the external power supply is a wearable patch.
